(19) United States
(12) Patent Application Publication
(10) Pub. No.: US 2023/0302041 A1
(43) Pub. Date: Sep. 28, 2023

(54) Competitive Self-Blocking with Unlabeled Manocept Imaging Agents

(71) Applicant: Navidea Biopharmaceuticals, Inc., Dublin, OH (US)

(72) Inventor: David A. Ralph, Columbus, OH (US)

(21) Appl. No.: 18/189,658

(22) Filed: Mar. 24, 2023

Related U.S. Application Data
(60) Provisional application No. 63/323,993, filed on Mar. 25, 2022.

(51) Int. Cl. A61K 31/721; A61K 51/06; A61P 13/12; A61P 1/00; A61P 17/00

(52) U.S. Cl. CPC A61K 31/721; A61K 51/065; A61P 13/12; A61P 1/00; A61P 17/00

(57) ABSTRACT

Disclosed is a method for increased target specificity of a mannosylated carbohydrate polymeric therapeutic and/or diagnostic compound by administering a blocking composition comprising a backbone and one or more C-type lectin receptor targeting moieties attached thereto with an effective amount of the mannosylated dextran therapeutic and/or diagnostic compound. The administration of the blocking compound results in higher localization of the mannosylated dextran therapeutic and/or diagnostic compounds to a desired target other than the liver and/or spleen compared to without the administration of the blocking compound. The molecular weight of the blocking composition backbone is between about 1 kDa and about 35 kDa.

### CROSS-REFERENCE TO RELATED APPLICATION(S)

This application claims the benefit under 35 U.S.C. § 119(e) to U.S. Provisional Application No. 63/323,993, filed Mar. 25, 2022, and entitled “Competitive Self-Blocking with Unlabeled Manocept Imaging Agents,” which is hereby incorporated by reference in its entirety for all purposes.

### BACKGROUND OF THE INVENTION

CD206, also known as macrophage mannose receptor (MMR), is a C-type lectin transmembrane protein that plays a crucial role in innate immune responses. It is primarily expressed on the surface of macrophages, dendritic cells, some classes of myeloid derived suppressor cells (MDSC) and mesangial cells of the kidney. CD206 binds to mannose-containing structures which are commonly found on the surface of pathogens, and facilitates their internalization and degradation. Macrophages are involved in various pathologies. Macrophage involved pathologies include cancer, atherosclerosis, and various autoimmune diseases, such as but not limited to, Crohn's disease, rheumatoid arthritis, systemic lupus erythematosus and multiple sclerosis. Macrophages and dendritic cells are also key components of innate immunity, which is the body's first line of defense against infectious diseases. In addition, macrophages and dendritic cells are antigen presenting cells that communicate with and regulate aspects of cells such as lymphocytes that are involved in adaptive immunity. As such, there is a growing interest in developing imaging agents that can selectively bind to CD206 and allow for the visualization and quantification of CD206 expression in vivo. Such agents would be useful in diagnosing and monitoring diseases associated with CD206 expression, as well as in evaluating the efficacy of therapies targeting CD206.

Mannosylated amine dextrans (MADs) have been shown to bind selectively to CD206 and have therefore been proposed as a targeting agent for CD206 imaging. MADs are further being developed as drug delivery vehicles for delivery of small molecule payloads and imaging agents targeted to tumor associate macrophages (TAMs) and other sites of macrophage involved pathologies, where CD206 expressing macrophages are abundant. Examples include U.S. patent application Ser. Nos. 16/041,673; 16/832,620; 16/773,512; and Ser. No. 16/997,648, all of which are hereby incorporated by reference in their entireties.

However, CD206 is also expressed by Kupffer cells. Kupffer cells are resident macrophage-like cells of the liver that occur in large numbers along the walls of sinusoid capillaries. Thus, Kupffer cells are in direct contact with the blood. There are also large numbers of CD206 expressing cells in the spleen that are separated from the blood flow by limited barriers. Another important class of CD206 expressing cell is comprised of the mesangial cells, which reside in the glomeruli of the kidneys. Mesangial cells are separated from the blood only by an incomplete layer of endothelial cells and an acellular membrane that allows molecules smaller than about the size of serum albumen (approximately 65,000 Daltons) to pass into the glomerular filtrate, thus affording mesangial cells largely unobstructed contact with smaller macromolecules in the blood, such as the MAD constructs described in this disclosure. Therefore, mannosylated dextran conjugates if administered directly (e.g. by intravenous injection) into the blood flow can exhibit off-target localization in the liver, spleen, and kidney, which can limit their imaging specificity and sensitivity, and in embodiments where the mannosylated dextran carries small molecule therapeutic payloads, can result in off-target delivery of payloads to these tissues.

To address this issue, various strategies have been proposed to minimize the off-target localization of mannosylated dextran conjugates. For example, the size and charge of the conjugates can be optimized to reduce their uptake by non-target cells and organs. Examples include U.S. patent application Ser. Nos. 17/039,409 and 18/164,296, all of which are hereby incorporated in reference in their entireties. However, there remains an ongoing need for additional solutions to block off-target localization to areas containing certain C-type lectin receptors and especially for the macrophage mannose receptor, CD206.

Accordingly, there is a need in the art for alternative means to synthesize carbohydrate polymeric constructs with differential localization potential to avoid off-target liver, spleen, and mesangial cell localization while promoting equal or greater localization to target tissues, such as tumors (TAMs, dendritic cells, and MDSC), and/or to sites of inflammation or infection.

### BRIEF SUMMARY OF THE INVENTION

Disclosed are methods of increasing target specificity of a mannosylated dextran therapeutic and/or diagnostic compound, methods of diagnosing and treating a disease, and a kit for the diagnosis or treatment of a subject in need thereof.

In Example 1, a method of increasing target specificity of a mannosylated carbohydrate polymeric therapeutic and/or diagnostic compound comprises (a) administering a blocking compound comprising a backbone and one or more C-type lectin receptor targeting moieties attached thereto, and (b) administering an effective amount of the mannosylated carbohydrate polymeric therapeutic and/or diagnostic compound comprising a carbohydrate polymeric backbone, one or more C-type lectin receptor targeting moieties, and one or more therapeutic, diagnostic, or theranostic agents attached thereto, wherein the blocking compound backbone is between about 1 kDa and about 35 kDa.

Example 2 relates to the method according to Example 1, wherein the mannosylated carbohydrate polymeric therapeutic and/or diagnostic compound comprises the compound of Formula (I):

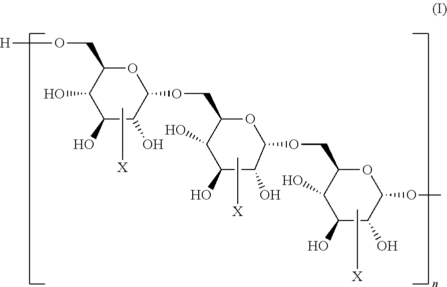

wherein
each X is independently H, L1-A, L1-Y-A, L2-R, or L3-Y, wherein each X is bound to any OH group;
each L1, L2, and L3 are independently leashes having the formula —(CH₂)ₚS(CH₂)_{q}—NH—,
wherein p and q are independently integers from 0 to 5;
each A independently comprises a therapeutic agent, a diagnostic agent, a theranostic agent, or H;
each Y independently comprises a chelating agent;
each R independently comprises a mannose-binding C-type lectin receptor targeting moiety or H;
and n is an integer greater than zero, wherein each unit of n may be the same or different; and
wherein at least one R comprises a mannose-binding C-type lectin receptor targeting moiety selected from the group consisting of mannose, fucose, and n-acetylglucosamine; and
wherein at least one A comprises a therapeutic agent, diagnostic agent, or theranostic agent.

Example 3 relates to the method according to Example 1, wherein the carbohydrate polymeric backbone is selected from the group consisting of dextran, cellulose, polyethylene glycol, and polypeptides.

Example 4 relates to the method according to Example 1, wherein the one or more C-type lectin receptor targeting moieties is attached to the blocking compound backbone via a leash having the formula —(CH₂)ₚS(CH₂)_{q}—NH—, wherein p and q are independently integers from 0 to 5.

Example 5 relates to the method according to Example 1, wherein the mannosylated dextran therapeutic and/or diagnostic compound dextran backbone is between about 1 kDa and about 50 kDa, and wherein the blocking compound backbone is between about 1 kDa and 30 kDa.

Example 6 relates to the method according to Example 1, wherein the blocking compound comprises a compound of Formula (II):

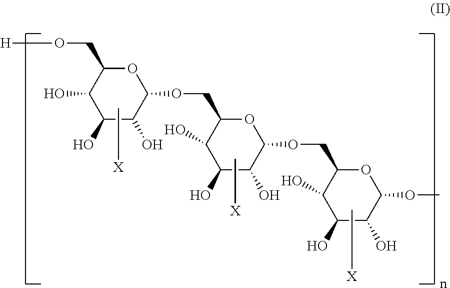

wherein
each X is independently H or L2-R, wherein each X is bound to any OH group;
each R independently comprises a mannose-binding C-type lectin receptor targeting moiety or H; each L2 is independently a leash having the formula —(CH₂)ₚS(CH₂)_{q}—NH—, wherein p and q are independently integers from 0 to 5;
n is an integer greater than zero, wherein each unit of n may be the same or different; and
wherein at least one R comprises a mannose-binding C-type lectin receptor targeting moiety selected from the group consisting of mannose, fucose, and n-acetylglucosamine.

Example 7 relates to the method according to Example 1, wherein the blocking compound does not contain a therapeutic agent, diagnostic agent, theranostic agent, or a detectable moiety.

Example 8 relates to the method according to Example 1, wherein the step of administering the blocking compound is followed by a time interval of from zero to about 60 minutes before the step of administering the mannosylated dextran therapeutic and/or diagnostic compound.

Example 9 relates to the method according to Example 1, wherein the blocking compound and the mannosylated dextran therapeutic and/or diagnostic compound are administered simultaneously.

Example 10 relates to the method according to Example 1, wherein the mannosylated dextran therapeutic and/or diagnostic compound has decreased localization to C-type lectin receptor cells in the liver and/or spleen relative to a subject administered a comparable dose of mannosylated dextran therapeutic and/or diagnostic compound without administration of the blocking compound.

Example 11 relates to the method according to Example 1, wherein the administration of the blocking compound results in higher localization to a desired target other than the liver and/or spleen compared to without the administration of the blocking compound.

Example 12 relates to the method according to Example 11, wherein the desired target comprises the kidneys, large intestine, and/or skin.

Example 13 relates to the method according to Example 1, wherein the effective dose of the mannosylated dextran therapeutic and/or diagnostic compound is lower than the effective does of the mannosylated dextran therapeutic and/or diagnostic compound without administration of the blocking compound.

Example 14 relates to the method according to Example 1, wherein the blocking compound preferentially binds to CD206 expressing cells in the liver and/or spleen.

Example 15 relates to the method according to Example 1, wherein the subject has been diagnosed with disease associated with the kidneys, large intestine, or skin.

Example 16 relates to the method according to Example 15, wherein the disease comprises renal failure, polycystic kidney disease, nephritis, Crohn's Disease, irritable bowel syndrome, Celiac disease, colon cancer, psoriasis, diabetic neuropathy, or a combination thereof.

Example 17 relates to the method according to Example 1, wherein the blocking agent is administered to a human subject in an amount of at least about 50 mg.

In Example 18, a method of diagnosing and treating a disease comprises (a) administering a blocking compound comprising a backbone and one or more C-type lectin receptor targeting moieties attached thereto, wherein the blocking compound backbone is from about 1 kDa to about 35 kDa, and (b) administering an effective amount of a mannosylated carbohydrate polymeric therapeutic and/or diagnostic compound comprising a carbohydrate polymeric backbone and one or more C-type lectin receptor targeting moieties and one or more therapeutic, diagnostic, or theranostic agents attached thereto, wherein the administration of the blocking compound increases target specificity of the mannosylated carbohydrate polymeric therapeutic and/or diagnostic compound.

Example 19 relates to the method according to Example 18, wherein the disease affects the kidneys, large intestine, or skin.

Example 20 relates to the method according to Example 19, wherein the disease comprises renal failure, polycystic kidney disease, nephritis, Chron's Disease, irritable bowel syndrome, Celiac disease, colon cancer, psoriasis, diabetic neuropathy, or a combination thereof.

Example 21 relates to the method according to Example 18, wherein the mannosylated carbohydrate polymeric therapeutic and/or diagnostic compound comprises the compound of Formula (I):

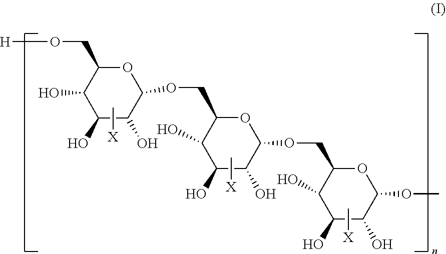

wherein
each X is independently H, L1-A, L1-Y-A, L2-R, or L3-Y, wherein each X is bound to any OH group;
each L1, L2, and L3 are independently leashes having the formula —(CH₂)ₚS(CH₂)_{q}—NH—,
wherein p and q are independently integers from 0 to 5;
each A independently comprises a therapeutic agent, a diagnostic agent, a theranostic agent, or H; each Y independently comprises a chelating agent;
each R independently comprises a mannose-binding C-type lectin receptor targeting moiety or H; and n is an integer greater than zero, wherein each unit of n may be the same or different; and
wherein at least one R comprises a mannose-binding C-type lectin receptor targeting moiety selected from the group consisting of mannose, fucose, and n-acetylglucosamine; and
wherein at least one A comprises a therapeutic agent, diagnostic agent, or theranostic agent.

Example 22 relates to the method according to Example 18, wherein the blocking compound comprises a compound of Formula (II):

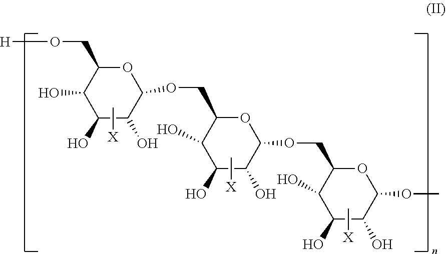

wherein
each X is independently H or L2-R, wherein each X is bound to any OH group;
each R independently comprises a mannose-binding C-type lectin receptor targeting moiety or H; each L2 is independently a leash having the formula —(CH₂)ₚS(CH₂)_{q}—NH—, wherein p and q are independently integers from 0 to 5;
n is an integer greater than zero, wherein each unit of n may be the same or different; and
wherein at least one R comprises a mannose-binding C-type lectin receptor targeting moiety selected from the group consisting of mannose, fucose, and n-acetylglucosamine.

Example 23 relates to the method according to Example 18, wherein the blocking compound backbone is from about 1 kDa to about 50 kDa, and the mannosylated dextran therapeutic and/or diagnostic compound dextran backbone is between about 1 kDa and about 30 kDa.

While multiple embodiments are disclosed, still other embodiments of the present disclosure will become apparent to those skilled in the art from the following detailed description, which shows and describes illustrative embodiments of the disclosure. Accordingly, the figures and detailed description are to be regarded as illustrative in nature and not restrictive.

### BRIEF DESCRIPTION OF THE FIGURES

FIG. 1A shows a bar graph of the biodistribution comparison of the tracer [68]Ga-MAN-DOTA-3.5 under non-competitive and competitive conditions. The error bars indicate standard deviation, where n=4 per group.

FIG. 1B shows a bar graph of the biodistribution comparison of the tracer [68]Ga-MAN-DOTA-10 under non-competitive and competitive conditions. The error bars indicate standard deviation, where n=4 per group.

FIG. 2 shows a bar graph showing the percent of localization with competition relative to without competition using [68] Ga MAN-DOTA-10 kDa.

Various embodiments of the present disclosure will be described in detail with reference to the figures. Reference to various embodiments does not limit the scope of the disclosure.

Figures represented herein are not limitations to the various embodiments according to the disclosure and are presented for exemplary illustration of the disclosure.

### DETAILED DESCRIPTION

It is to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “substantially free” refers to compositions completely lacking the component or having such a small amount of the component that the component does not affect the performance of the composition. The component may be present as an impurity or as a contaminant and shall be less than 0.5 wt-%. In another embodiment, the amount of the component is less than 0.1 wt-% and in yet another embodiment, the amount of component is less than 0.01 wt-%.

The term “weight percent,” “wt-%,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt-%,” etc.

The compounds, compositions, methods, kits, and systems of the present disclosure may comprise, consist essentially of, or consist of the components and ingredients of the present disclosure as well as other ingredients described herein. As used herein, “consisting essentially of” means that the compounds, compositions, methods, kits, and systems may include additional steps, components or ingredients, but only if the additional steps, components or ingredients do not materially alter the basic and novel characteristics of the claimed compounds, compositions, methods, kits, and systems.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH2CH2O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A1,” “A2,” “A3,” and “A4” are used herein as generic symbols to represent various specific substituents. These symbols can be any substituent, not limited to those disclosed herein, and when they are defined to be certain substituents in one instance, they can, in another instance, be defined as some other substituents.

“R1,” “R2,” “R3,” “Rn,” where n is an integer, as used herein can, independently, possess one or more of the groups listed above. For example, if R1 is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the disclosure may contain “optionally substituted” moieties. In general, the term “substituted,” whether preceded by the term “optionally” or not, means that one or more hydrogens of the designated moiety are replaced with a suitable substituent.

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. In is also contemplated that, in certain aspects, unless expressly indicated to the contrary, individual substituents can be further optionally substituted (i.e., further substituted or unsubstituted).

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific embodiment or combination of embodiments of the methods of the disclosure.

As used herein, the term “pharmaceutically acceptable carrier” or “carrier” refers to sterile aqueous or nonaqueous solutions, colloids, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, cancer of the small intestine, and cancer of the esophagus. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. In some embodiments, the present methods can be used to treat a subject having an epithelial cancer, e.g., a solid tumor of epithelial origin, e.g., lung, breast, ovarian, prostate, renal, pancreatic, or colon cancer.

As used herein, the term “subject” refers to the target of administration, e.g., an animal. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more cancer disorders prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with cancer” means having been subjected to a physical examination by a person of skill, for example, a physician, and found to have a condition that can be diagnosed or treated by a compound or composition that can reduce tumor size or slow rate of tumor growth. A subject having cancer, tumor, or at least one cancer or tumor cell, may be identified using methods known in the art. For example, the anatomical position, gross size, and/or cellular composition of cancer cells or a tumor may be determined using contrast-enhanced MRI or CT. Additional methods for identifying cancer cells can include, but are not limited to, ultrasound, bone scan, surgical biopsy, and biological markers (e.g., serum protein levels and gene expression profiles). An imaging solution comprising a cell-sensitizing composition of the present disclosure may be used in combination with MRI or CT, for example, to identify cancer cells.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, administration to specific organs through invasion, intramuscular administration, intratumoral administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

Effective dosages may be estimated initially from in vitro assays. For example, an initial dosage for use in animals may be formulated to achieve a circulating blood or serum concentration of active compound that is at or above an IC50 of the particular compound as measured in an in vitro assay. Calculating dosages to achieve such circulating blood or serum concentrations, taking into account the bioavailability of the particular active agent, is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamagon Press, which is hereby incorporated by reference in its entirety, and the references cited therein.

The phrase “anti-cancer composition” can include compositions that exert antineoplastic, chemotherapeutic, antiviral, antimitotic, antitumorgenic, anti-angiogenic, anti-metastatic and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agents available in commercial use, in clinical evaluation and in pre-clinical development, which could be included in this application by combination drug chemotherapy. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids.

The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

The term “theranostic” as used herein refers to therapy which combines diagnostic and therapeutic capabilities in a single agent.

“Tilmanocept” refers to a non-radiolabeled precursor of the LYMPHOSEEK® diagnostic agent. Tilmanocept is a mannosylaminodextran (MAD). It has a dextran backbone to which a plurality of amino-terminated leashes (—O(CH₂)₃S(CH₂)₂NH₂) are attached to the core glucose elements. In addition, mannose moieties are conjugated to amino groups of a number of the leashes, and the chelator diethylenetriamine pentaacetic acid (DTPA) may be conjugated to the amino group of other leashes not containing the mannose. Tilmanocept generally, has a dextran backbone, in which a plurality of the glucose residues comprises an amino-terminated leash:

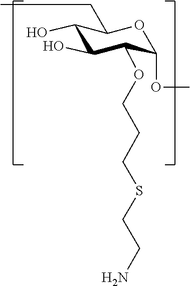

the mannose moieties are conjugated to the amino groups of the leash via an amidine linker:

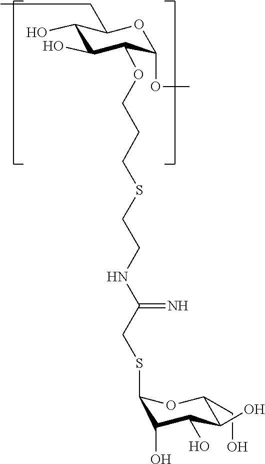

the chelator diethylenetriamine pentaacetic acid (DTPA) is conjugated to the amino groups of the leash via an amide linker:

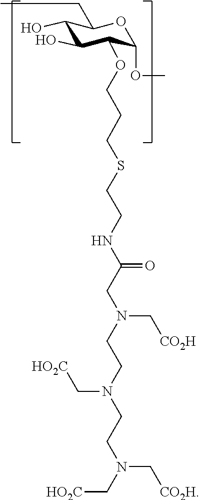

Tilmanocept has the chemical name dextran 3-[(2-aminoethyl)thio]propyl 17-carboxy-10,13,16-tris(carboxymethyl)-8-oxo-4-thia-7,10,13,16-tetraazaheptadec-1-yl 3-[[2-[[1-imino-2-(D-mannopyranosylthio)ethyl]amino]ethyl]thio]propyl ether complexes, and tilmanocept Tc99m has the following molecular formula: [C₆H₁₀O₅]ₙ·(Cl₉H₂₈N₄O₉S^{99m}Tc)_{b}·(C₁₃H₂₄N₂O₅S₂)_{c}·(C₅H₁₁NS)ₐ and contains 3-8 conjugated DTPA molecules (b); 12-20 conjugated mannose molecules (c); and 0-17 amine side chains (a) remaining free. Tilmanocept has the following general structure:

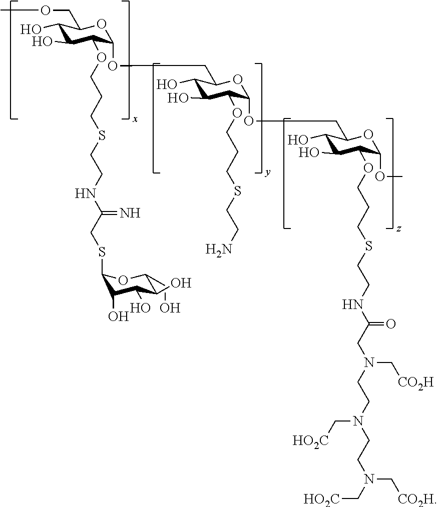

Certain of the glucose moieties may have no attached amino-terminated leash.

The present disclosure describes compositions of matter and methods for their use in increasing target specificity of a mannosylated carbohydrate polymeric therapeutic and/or diagnostic compound. Provided are a series of C-type lectin receptor targeting agents utilizing mannosylated carbohydrate polymeric compounds, such as, mannosylated amine dextrans (MAD), conjugated to one or more C-type lectin targeting moieties for use as a diagnostic, therapeutic, or theranostic construct. As described throughout the present disclosure, the term “diagnostic and/or therapeutic compound” will refer to diagnostic compounds, therapeutic compounds, or theranostic compounds. Further provided are competitive blocking compounds comprising a carbohydrate polymeric backbone and one or more C-type lectin targeting moieties attached thereto. In embodiments, the competitive blocking compounds are substantially similar to the mannosylated carbohydrate polymeric therapeutic and/or diagnostic compounds, minus the labeling agent and/or therapeutic agent. In further embodiments, the blocking compounds are substantially similar to the mannosylated carbohydrate polymeric therapeutic and/or diagnostic compounds, minus any chelating agents.

Beneficially, the competitive blocking compounds bind to C-type lectin receptor expressing cells, such as, but not limited to, the liver and spleen without proportionally diminishing the abilities of the mannosylated carbohydrate polymeric therapeutic and diagnostic compounds from binding to C-type lectin receptor expressing cells that have aggregated at sites of pathological processes. In further embodiments, the disclosure enables robust localization of the mannosylated carbohydrate polymeric therapeutic and diagnostic compounds to sites of pathological processes while reducing or eliminating localization to off-target sites such as the liver and spleen. Off-target localization has undesirable and/or dose limiting consequences, therefore, the blocking compounds disclosed beneficially provide competitive blocking to reduce the accumulation of the mannosylated carbohydrate polymeric therapeutic and diagnostic compounds (or other diagnostic imaging and/or therapeutic agents) in off-target sites, while permitting localization of the mannosylated carbohydrate polymeric therapeutic and diagnostic compounds to pathological lesions. In some aspects, the competitive blocking compounds disclosed further provide for significantly increased localization of the mannosylated carbohydrate polymeric therapeutic and diagnostic compounds to specific organs and tissues containing pathological lesions, including, but not limited to, the kidneys, the large intestine, and skin.

In certain aspects, the present disclosure further provide for methods of diagnosing and treating diseases. In part, due to the ability of the disclosed blocking compounds to increase target specificity of the mannosylated carbohydrate polymeric therapeutic and diagnostic compounds, the combination of the blocking compound and mannosylated carbohydrate polymeric therapeutic and diagnostic compounds disclosed herein further provide for the ability to treat and diagnose diseases by improving the efficacy of the mannosylated carbohydrate polymeric therapeutic and diagnostic compounds to localize to pathological lesions that require imaging or treating.

Mannosylated Carbohydrate Polymeric Therapeutic and/or Diagnostic Compounds

In certain aspects, compounds disclosed herein employ a carrier construct comprising a carbohydrate polymeric backbone having conjugated thereto mannose-binding C-type lectin receptor targeting moieties (e.g., mannose, fucose, and N-acetylglucosamine) to deliver one or more active therapeutic agents or diagnostic agents. Examples of such constructs include mannosylamino dextrans (MAD), which comprise a dextran backbone having mannose molecules conjugated to glucose residues of the backbone and having an active pharmaceutical ingredient conjugated to glucose residues of the backbone. Tilmanocept is a specific example of an MAD. A tilmanocept derivative that is tilmanocept without DTPA conjugated thereto is a further example of an MAD. In certain aspects, the carbohydrate polymeric therapeutic and/or diagnostic compound disclosed herein comprise a charged compound. In exemplary embodiments, the carbohydrate polymeric therapeutic and/or diagnostic compounds are polycationic and synthesized to have a positive charge.

In certain implementations, the disclosure provides for constructs having a carbohydrate polymeric backbone selected from the group consisting of dextran, cellulose, polyethylene glycol, and polypeptides. In further implementations, the carbohydrate polymeric backbone is dextran. The disclosure further provides for compounds comprising a dextran-based moiety or backbone having one or more mannose-binding C-type lectin receptor targeting moieties and one or more therapeutic and/or diagnostic agents attached thereto. The dextran-based moiety generally comprises a dextran backbone similar to that described in U.S. Pat. No. 6,409,990 (the ′990 patent), which is incorporated herein by reference. Thus, the backbone comprises a plurality of glucose moieties (i.e., residues) primarily linked by α-1,6 glycosidic bonds. Other linkages such as α-1,4 and/or α-1,3 bonds may also be present. In some embodiments, not every backbone moiety is substituted. In some embodiments, mannose-binding C-type lectin receptor targeting moieties are attached to between about 10% and about 50% of the glucose residues of the dextran backbone, or between about 20% and about 45% of the glucose residues, or between about 25% and about 40% of the glucose residues. In some embodiments, the dextran backbone has a molecular weight (Mw) of between about 1 and about 500 kDa, about 1 and about 450 kDa, about 1 and about 400 kDa, about 1 and about 350 kDa, about 1 and about 300 kDa, about 1 and about 250 kDa, about 1 and about 200 kDa, about 1 and about 150 kDa, about 1 and about 100 kDa, or about 1 and about 50 kDa. In additional embodiments, the backbone has a molecular weight of between about 1 and about 20 kDa, about 1 and about 15 kDa, about 1 and about 10 kDa, about 1 and about 5 kDa, while in other embodiments the backbone has a molecular weight of between about 5 and about 40 kDa. In still other embodiments, the backbone has a molecular weight of between about 8 and about 15 kDa, such as about 10 kDa. While in other embodiments the backbone has a molecular weight of between about 1 and about 5 kDa, such as about 3.5 kDa.

According to further aspects, the mannose-binding C-type lectin receptor targeting moiety is selected from, but not limited to, mannose, galactose, fucose, N-acetylgalactosamine (GalNAc), N-acetylglucosamine (GlcNAc), and/or glucuronic acid. In some embodiments, the targeting moieties are attached to between about 10% and about 80% of the glucose residues of the dextran backbone, or between about 20% and about 75% of the glucose residues, or between about 25% and about 70% of the glucose residues. Molecular weights referenced herein, as well as the number and degree of conjugation of receptor substrates, leashes, and diagnostic/therapeutic moieties attached to the dextran backbone refer to average amounts for a given quantity of carrier molecules, since the synthesis techniques will result in some variability.

According to certain embodiments, the one or more mannose-binding C-type lectin receptor targeting moieties and one or more therapeutic and/or diagnostic agents are attached to the dextran-based moiety by way of a leash. The leash may be attached to from about 30% to about 100% of the backbone glucose moieties or from about 35% to about 90%. The leashes may be the same or different. In some embodiments, the leash is an amino-terminated leash. In some embodiments, the leashes may comprise the formula —(CH₂)ₚS(CH₂)_{q}—NH—, wherein p and q are integers from 0 to 5. In further embodiments, the leash comprises the formula —(CH₂)₃S(CH₂)₂NH—. In embodiments where the leash is not attached to a mannose-binding C-type lectin receptor targeting moiety, the leash may comprise the formula —(CH₂)ₚS(CH₂)_{q}—NH₂, wherein p and q are integers from 0 to 5. In some embodiments, the leashes may comprise —O(CH₂)₃S(CH₂)₂NH—. In some embodiments, the leash may be a chain of from 1 to 20 member atoms selected from carbon, oxygen, sulfur, nitrogen and phosphorus. The leash may be a straight chain or branched. The leash may also be substituted with one or more substituents including, but not limited to, halo groups, perfluoroalkyl groups, perfluoroalkoxy groups, alkyl groups, such C1-4 alkyl, alkenyl groups, such as C1-4 alkenyl, alkynyl groups, such as C1-4 alkynyl, hydroxy groups, oxo groups, mercapto groups, alkylthio groups, alkoxy groups, nitro groups, azidealkyl groups, aryl or heteroaryl groups, aryloxy or heteroaryloxy groups, aralkyl or heteroaralkyl groups, aralkoxy or heteroaralkoxy groups, HO—(C═O)— groups, heterocylic groups, cycloalkyl groups, amino groups, alkyl- and dialkylamino groups, carbamoyl groups, alkylcarbonyl groups, alkylcarbonyloxy groups, alkoxycarbonyl groups, alkylaminocarbonyl groups, dialkylamino carbonyl groups, arylcarbonyl groups, aryloxycarbonyl groups, alkylsulfonyl groups, arylsulfonyl groups, —NH—NH₂; ═N—H; ═N-alkyl; SH; —S-alkyl; NH—C(O); —NH—C(═N)— and the like. As would be apparent to one skilled in the art, other suitable leashes are possible. In other embodiments, the mannose-binding C-type lectin targeting moieties may be conjugated to the amino groups of the amino terminated leash via an amidine and/or amide linker.

In some embodiments, the one or more therapeutic agents is attached via a biodegradable linker. In some embodiments, the biodegradable linker comprises a pH sensitive moiety, such as a hydrazone. At lower (more acidic) pH, hydrazone linkers spontaneously hydrolyze at increasing rates as pH decreases. When a mannosylated carbohydrate polymeric binds to CD206, it is internalized to endosomes which become increasingly acidified over time, thereby releasing the therapeutic agent payloads intracellularly.

In certain aspects, a chelating agent may be attached to or incorporated into the disclosed mannosylated carbohydrate polymeric therapeutic and/or diagnostic compounds. In embodiments, the chelating agent enables labeling of the mannosylated carbohydrate polymeric diagnostic (or imaging) compounds with radioactive metal ions. In further embodiments, the chelating agent may be used to chelate a therapeutic agent. Exemplary chelators include but are not limited to diethylenetriamine pentaacetic acid (DTPA), tetraazacyclododecanetetraacetic acid (DOTA), Triethylenetetramine (TETA), {4-[2-(bis-carboxymethylamino)-ethyl]-7carboxymethyl-[1,4,7]-triazonan-1-yl}-acetic acid (NETA), 1,4,7-triazacyclononane-N,N′,N″-triacetic acid (NOTA), mercaptoacetylglycylglycyl-glycine (MAG3), dimercaptosuccinic acid, diphenylethylene diamine, porphyrin, iminodiacetic acid, and ethylenediaminetetraacetic acid (EDTA). In embodiments, the chelating agent may be attached to the carbohydrate polymeric backbone via an amino-terminated leash. The chelating agent may be further bound to the amino-terminated leash via an amide linker. In some embodiments, the chelating agent attached to the carbohydrate polymeric backbone is not further attached to any diagnostic and/or therapeutic agents. In further embodiments, the mannosylated carbohydrate polymeric therapeutic and/or diagnostic compounds may not include a chelating agent.

According to embodiments, the therapeutic agent is a cytotoxic agent. In further embodiments, the therapeutic agent is an anti-cancer agent. In further embodiments, the therapeutic agent may include, but is not limited to, doxorubicin, paclitaxel, bisphosphonates, a metal ion, and anti-inflammatory agents such as dexamethasone. In some aspects, the metal ion may include copper, iron, arsenic, antimony silver, cadmium, gallium or gadolinium. In further aspects, the therapeutic agent may further comprise a radioisotope, including but not limited to, ^{99m}Tc, ^{210/212/213/214}Bi ¹³¹Ba, ¹⁴⁰Ba, ^{11/14}C, ⁵¹Cr, ^{67/68}Ga, ¹⁵³Gd, ^{88/90/91}Y, ^{123/124/125/131}I, ^{111/115m}In, ¹⁸F, ¹³N, ¹⁰⁵Rh ¹⁵³Sm, ⁶⁷Cu, ¹⁶⁶Ho, ¹⁷⁷Lu, ²²³Ra, ⁶²Rb, ^{186/188}Re, ^{32/33}P, ^{46/47}Sc, ^{72/75}Se, ³⁵S, ⁸⁹Sr, ¹⁸²Ta, ^{123m}Te, ¹²⁷Te, ¹²⁹Te, ¹³²Te, ⁶⁵Zn, ^{89/95}Zr; or other chelateable isotope(s). In embodiments, the concentration of the radioisotope may be present in an amount of at least about 100 mCi (millicurie).

According to further embodiments, any of a variety of detectable moieties can be attached to the carrier molecule, directly or indirectly, for a variety of purposes. As used herein, the term “detectable moiety,” “diagnostic moiety,” or “diagnostic agent” (which these terms may be used interchangeably) means an atom, isotope, or chemical structure which is: (1) capable of attachment to the carrier molecule; (2) non-toxic to humans; and (3) provides a directly or indirectly detectable signal, particularly a signal which not only can be measured but whose intensity is related (e.g., proportional) to the amount of the detectable moiety. The signal may be detected by any suitable means, including spectroscopic, electrical, optical, magnetic, auditory, radio signal, or palpation detection means as well as by the measurement processes described herein.

Suitable detectable moieties include, but are not limited to radioisotopes (radionuclides), fluorophores, chemiluminescent agents, bioluminescent agents, magnetic moieties (including paramagnetic moieties), metals (e.g., for use as contrast agents), RFID moieties, enzymatic reactants, colorimetric release agents, dyes, and particulate-forming agents. In some aspects, the radioisotopes may be present in an amount of from about 0.01 mCi to about 40 mCi, about 0.05 mCi to about 35 mCi, or about 0.1 mCi to about 30 mCi.

By way of specific example, suitable diagnostic moieties include, but are not limited to:
contrast agents suitable for magnetic resonance imaging (MRI), such as gadolinium (Gd3+), paramagnetic and superparamagnetic materials such as superparamagnetic iron oxide;
contrast agents suitable for computed tomographic (CT) imaging, such as iodinated molecules, ytterbium and dysprosium;
radioisotopes suitable for scintigraphic imaging (or scintigraphy) such as ^{99m}Tc, ^{210/212/213/214}Bi ¹³¹Ba, ¹⁴⁰Ba, ^{11/14}C, ⁵¹Cr, ^{67/68}Ga, ¹⁵³Gd, ^{88/90/91}Y, ^{123/124/125/131}I, ^{111/115m}In, ¹⁸F, ¹³N, ¹⁰⁵Rh, ¹⁵³Sm, ⁶⁷Cu ¹⁶⁶Ho, ¹⁷⁷Lu, ²²³Ra, ⁶²Rb, ^{186/188}Re, ^{32/33}P, ^{46/47}Sc, ^{72/75}Se, ³⁵S, ⁸⁹Sr, ¹⁸²Ta, ^{123m}Te, ¹²⁷Te, ¹²⁹Te, ¹³²Te, ⁶⁵Zn, ^{89/95}Zr; or other chelateable isotope(s);
gamma-emitting agents suitable for single-photon emission computed tomography (SPECT), such as ^{99m}Tc, ¹¹¹In, and ¹²³I;
dyes and fluorescent agents suitable for optical imaging; and
agents suitable for positron emission tomography (PET) such as ¹⁸F.

A diagnostic moiety can be attached to the carrier molecule in a variety of ways, such as by direct attachment or using a chelator attached to a carrier molecule. In some embodiments, diagnostic moieties can be attached using leashes attached to a carrier backbone. In some embodiments, the chelator can be conjugated to an amino group of one or more leashes and can be used to bind the diagnostic moiety thereto. It should be noted that in some instances, glucose moieties of the carbohydrate polymeric backbone may have no attached leash. Certain embodiments may include a single type of diagnostic moiety or a mixture of different diagnostic moieties. For example, an embodiment of a compound disclosed herein may comprise a contrast agent suitable for MRI and a radioisotope suitable for scintigraphic imaging, and further combinations of the diagnostic moieties described herein. In embodiments, after administration of a diagnostic or imaging compound to an animal or human subject, the radioactive metal ions enable single-photon emission computed tomography (SPECT) or positron emission tomography (PET) imagining of anatomical locations or sites where the labeled imaging agents are present.

In certain aspects, the disclosed compounds are present in the form of a pharmaceutically acceptable carrier.

According to certain embodiments, the disclosed carbohydrate polymeric therapeutic and/or diagnostic compound is a compound of Formula (I):

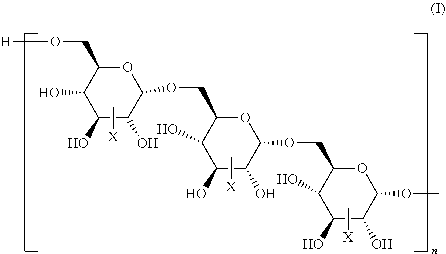

wherein
each X is independently H, L1-A, L1-Y-A, L2-R, or L3-Y, wherein each X is bound to any OH group;
each L1, L2, and L3 are independently leashes;
each A independently comprises a therapeutic agent, a diagnostic agent, a theranostic agent, or H; each Y independently comprises a chelating agent;
each R independently comprises a mannose-binding C-type lectin receptor targeting moiety or H;
and n is an integer greater than zero, wherein each unit of n may be the same or different; and
wherein at least one R comprises a mannose-binding C-type lectin receptor targeting moiety selected from the group consisting of mannose, fucose, and n-acetylglucosamine; and
wherein at least one A comprises a therapeutic agent, diagnostic agent, or theranostic agent.

In certain embodiments, each of L1, L2, and L3 comprise the formula —(CH₂)ₚS(CH₂)_{q}—NH—, wherein p and q are independently integers from 0 to 5.

According to further embodiments, at least one of L1, L2, or L3 is a C2-12 hydrocarbon chain optionally interrupted by up to three heteroatoms selected from the group consisting of O, S and N.

In some aspects, n is an integer greater than zero. In other aspects, n is an integer greater than 1. In further aspects, n may be an integer between 1 and about 50, between about 5 and about 40, or between about 5 and about 30. As would be understood by those skilled in the art, each unit of n may be the same or may be different. As each X may independently be H, L1-A, L1-Y-A, L2-R, or L3-Y, each unit of n may consist of any combination of X selected from H, L1-A, L1-Y-A, L2-R, or L3-Y.

By way of example only, the mannosylated carbohydrate polymeric therapeutic and diagnostic compound may comprise a unit n having the following formula shown in formula (Ia):

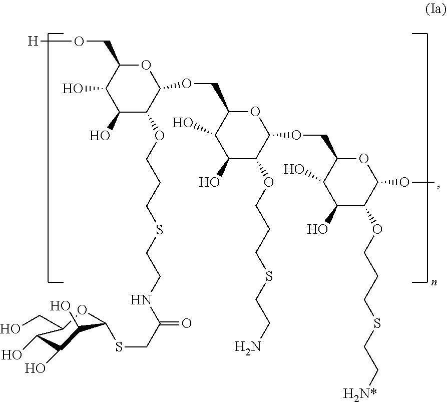

wherein the * indicates the point at which the therapeutic agent or diagnostic agent is attached. In certain embodiments, the therapeutic agent or diagnostic agent is attached at the end of an amino-terminated leash with or without a linker. In further embodiments, a chelating agent may be attached at the end of the amino-terminated leash with or without a linker. The chelating agent may optionally be further attached to a diagnostic agent, therapeutic agent, or theranostic agent.

### Blocking Compounds

In certain aspects, the blocking compounds disclosed herein comprise a compound having the capability to bind to C-type lectin receptors. In exemplary embodiments, the blocking compounds may be substantially similar to or the same as the mannosylated carbohydrate polymer therapeutic and/or diagnostic compound, however, without any labeling agents or therapeutic agents.

In additional aspects, the blocking compounds may employ a carrier construct comprising a polymeric (e.g. carbohydrate) backbone having conjugated thereto mannose-binding C-type lectin receptor targeting moieties (e.g. mannose) to preferentially bind to CD206, and at least one leash. In certain implementations, the carbohydrate polymeric backbone is selected from the group consisting of dextran, cellulose, polyethylene glycol, and polypeptides. In further implementations, the carbohydrate polymeric backbone is dextran. In exemplary embodiments, the mannose-binding C-type lectin receptor targeting moieties bind to CD206 expressed in the liver and/or spleen. Examples of such constructs include mannosylamino dextrans (MAD), which comprise a dextran backbone having mannose molecules conjugated to glucose residues of the backbone. In alternative embodiments, the blocking composition backbone may be comprised of any polymer suitable for safe administration to a subject and suitable for conjugation of C-type lectin receptor targeting moieties (with or without a leash). Examples include, but are not limited to cellulose, hyaluronic acid, chitosan, pullulan, and various polypeptides.

In certain aspects, the molecular weight (Mw) of the blocking compound can be varied and controlled by altering the molecular weight (Mw) of the starting dextran backbone on which it is constructed. In certain aspects, the dextran backbone of the blocking compound has a molecular weight of between about 1 kDa and about 35 kDa, about 1 kDa and about 30 kDa, about 1 kDa and about 25 kDa, about 1 and about 20 kDa, about 1 and about 15 kDa, about 1 and about 10 kDa, or about 1 and about 5 kDa. In still other embodiments, the backbone has a molecular weight of between about 2 kDa and about 15 kDa, such as about 10 kDa. While in other embodiments the backbone has a molecular weight of between about 1 and about 5 kDa, such as about 3.5 kDa. The molecular weight (Mw) of the various blocking compounds may be smaller than, about the same, or greater than the molecular weight (Mw) of the mannosylated carbohydrate polymeric therapeutic and/or diagnostic compounds disclosed herein.

In some embodiments, the mannose-binding C-type lectin receptor targeting moiety is selected from, but not limited to, mannose, galactose, fucose, N-acetylgalactosamine (GalNAc), N-acetylglucosamine (GlcNAc), and/or glucuronic acid. In some embodiments, the targeting moieties are attached to between about 10% and about 100% of the available residues of the blocking compound backbone, or between about 20% and about 90% of the residues, or between about 25% and about 80% of the residues. In some embodiments, the C-type lectin receptor targeting moieties are attached to about 100% of the available residues of the blocking compound backbone.

According to certain embodiments, the one or more mannose-binding C-type lectin receptor targeting moieties are attached to the backbone by way of a leash. In some embodiments, the leash is an amino-terminated leash. In certain embodiments, the mannose-binding C-type lectin targeting moieties are attached to between about 15% and about 100%, between about 17% and about 65%, or about 20% and about 60% of the glucose residues via the amino terminated leashes. In further embodiments, the mannose-binding C-type lectin targeting moieties are attached to up to about 60%, up to about 70%, up to about 80%, up to about 90%, or up to about 100% of the glucose residues via the amino terminated leashes. In certain aspects, the percentages may vary depending on the size of the carbohydrate polymer backbone.

In aspects, the leash may be attached to from about 30% to about 100% of the backbone moieties, or from about 70% to about 90% of the backbone moieties. The leashes may be the same or different. In some embodiments, the leashes may comprise the formula —(CH₂)ₚS(CH₂)_{q}—NH—, wherein p and q are integers from 0 to 5. In further embodiments, the leash comprises the formula —(CH₂)₃S(CH₂)₂NH—. In embodiments where the leash is not attached to a mannose-binding C-type lectin receptor targeting moiety, the leash may comprise the formula —(CH₂)ₚS(CH₂)_{q}—NH₂, wherein p and q are integers from 0 to 5.

In some embodiments, the leash may be a chain of from 1 to 20 member atoms selected from carbon, oxygen, sulfur, nitrogen and phosphorus. The leash may be a straight chain or branched. The leash may also be substituted with one or more substituents including, but not limited to, halo groups, perfluoroalkyl groups, perfluoroalkoxy groups, alkyl groups, such C1-4 alkyl, alkenyl groups, such as C1-4 alkenyl, alkynyl groups, such as C1-4 alkynyl, hydroxy groups, oxo groups, mercapto groups, alkylthio groups, alkoxy groups, nitro groups, azidealkyl groups, aryl or heteroaryl groups, aryloxy or heteroaryloxy groups, aralkyl or heteroaralkyl groups, aralkoxy or heteroaralkoxy groups, HO—(C═O)— groups, heterocylic groups, cycloalkyl groups, amino groups, alkyl- and dialkylamino groups, carbamoyl groups, alkylcarbonyl groups, alkylcarbonyloxy groups, alkoxycarbonyl groups, alkylaminocarbonyl groups, dialkylamino carbonyl groups, arylcarbonyl groups, aryloxycarbonyl groups, alkylsulfonyl groups, arylsulfonyl groups, —NH—NH2; ═N—H; ═N-alkyl; SH; —S-alkyl; NH—C(O); —NH—C(═N)— and the like. As would be apparent to one skilled in the art, other suitable leashes are possible. In certain alternative embodiments, the targeting moiety is attached directly to the backbone without use of a leash. In other embodiments, the mannose-binding C-type lectin targeting moieties may be conjugated to the amino groups of the amino terminated leash via an amidine and/or amide linker.

In certain aspects, a chelating agent may optionally be attached to or incorporated into the disclosed blocking compounds. Exemplary chelators include but are not limited to diethylenetriamine pentaacetic acid (DTPA), tetraazacyclododecanetetraacetic acid (DOTA), triethylenetetramine (TETA), {4-[2-(bis-carboxymethylamino)-ethyl]-7carboxymethyl-[1,4,7]-triazonan-1-yl}-acetic acid (NETA), 1,4,7-triazacyclononane-N,N′,N″-triacetic acid (NOTA), mercaptoacetylglycylglycyl-glycine (MAG3), dimercaptosuccinic acid, diphenylethylene diamine, porphyrin, iminodiacetic acid, and ethylenediaminetetraacetic acid (EDTA). In embodiments, the chelating agent may be attached to the carbohydrate polymeric backbone via an amino-terminated leash. The chelating agent may be further bound to the amino-terminated leash via an amide linker. In embodiments, the blocking compounds comprise a chelating agent but do not contain any detectable moieties, such as labeling agents, or any therapeutic agents conjugated thereto. In preferred embodiments, the blocking compounds do not comprise a chelating agent.

According to certain embodiments, blocking compound comprises the compound of Formula (II):

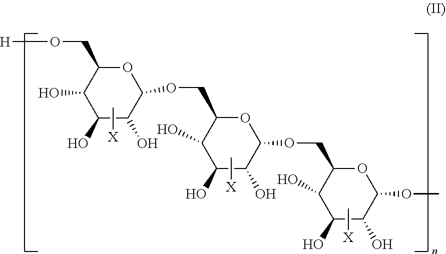

wherein
each X is independently H or L2-R, wherein each X is bound to any OH group;
each R independently comprises a mannose-binding C-type lectin receptor targeting moiety or H;
each L2 is independently a leash;
n is an integer greater than zero, wherein each unit of n may be the same or different.

In some embodiments, at least one R of formula (II) comprises a mannose-binding C-type lectin receptor targeting moiety selected from the group consisting of mannose, galactose, fucose, N-acetylgalactosamine (GalNAc), N-acetylglucosamine (GlcNAc), and/or glucuronic acid.

In some aspects, the leashes may be any leash as further described herein. In some embodiments, the L2 leash is an amino terminated leash, wherein the amino terminated leash may comprise the formula —(CH₂)ₚS(CH₂)_{q}—NH—, wherein p and q are integers from 0 to 5.

In some aspects, n is an integer greater than zero. In other aspects, n is an integer greater than 1. In further aspects, n may be an integer between 1 and about 50, between about 5 and about 40, or between about 5 and about 30. As would be understood by those skilled in the art, each unit of n may be the same or may be different. As each X may independently be H or L2-R, each unit of n may consist of any combination of X selected from H or L2-R.

### Therapeutic Compositions

According to certain embodiments, the disclosed mannosylated carbohydrate polymeric therapeutic and/or diagnostic compounds are further provided in a therapeutic composition. In some aspects, the mannosylated carbohydrate polymeric therapeutic and/or diagnostic compounds are provided with the blocking compounds in a therapeutic composition. In other aspects, the blocking compound is provided as a therapeutic composition separate from the mannosylated carbohydrate polymeric therapeutic and/or diagnostic compound provided as a therapeutic composition. In an aspect, the compositions may optionally further comprise a pharmaceutically acceptable carrier.

According to certain embodiments, the disclosed compounds (e.g., the mannosylated carbohydrate polymeric therapeutic and/or diagnostic compound, and/or the blocking compound) or a pharmaceutically acceptable salt of the compounds, can include a pharmaceutically acceptable carrier. The disclosed compounds, or pharmaceutically acceptable salts thereof, can also be included in pharmaceutical compositions in combination with one or more other therapeutically active compounds. In an aspect, the mannosylated carbohydrate polymeric therapeutic and/or diagnostic compound is administered with or shortly after the administration of a blocking compound. In additional aspects, the mannosylated carbohydrate polymeric therapeutic and/or diagnostic compound is administered without the use of a blocking compound. In embodiments, the pharmaceutically acceptable carrier employed may be, for example, a solid, liquid, or gas. Examples of solid carriers include lactose, mannitol, microcrystalline cellulose, terra alba, sucrose, talc, gelatin, agar, pectin, acacia, magnesium stearate, and stearic acid. Examples of liquid carriers are sugar syrup, peanut oil, olive oil, ethanol, propylene glycol, and water. Examples of gaseous carriers include carbon dioxide, nitrogen, and compressed air.

In preparing the compositions into a dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. In further aspects, aerosol carriers such as sugars comprising glucose, fructose, mannitol, sucrose, lactose, and cellulose, propellants, liquid carriers, and gaseous carriers may be utilized to form preparations suitable for inhalation. In certain aspects, the compositions are administered intravenously, intraperitoneally, intramuscularly, orally, subcutaneously, intra-tumorally or transdermally. In preferred embodiments, the compositions are administered intravenously.

In embodiments, the composition may include the blocking compound and the mannosylated carbohydrate polymeric therapeutic and/or diagnostic compound in any therapeutically effective amount as would be appreciated by those skilled in the art. In further embodiments, the blocking compound may be administered at a concentration or dose of at least 50 mg. In further embodiments, the amount of blocking compound will be administered to provide a molar excess of the blocking compound compared to the mannosylated carbohydrate polymeric therapeutic and/or diagnostic compound.

### Methods of Use

According to certain embodiments, disclosed is a method of increasing target specificity of a mannosylated carbohydrate polymeric therapeutic and/or diagnostic compound by administering a blocking compound and an effective amount of the mannosylated carbohydrate polymeric therapeutic and/or diagnostic compound. In certain aspects, disclosed is a method for increasing target specificity of a mannosylated carbohydrate polymeric therapeutic and/or diagnostic compound by administering a blocking compound comprising a backbone, and one or more C-type lectin receptor targeting moieties attached thereto; and administering an effective amount of the mannosylated carbohydrate polymeric therapeutic and/or diagnostic compound comprising a carbohydrate polymeric backbone, one or more C-type lectin receptor targeting moieties, and one or more therapeutic and/or diagnostic agents attached thereto. In embodiments, the blocking compound backbone is between about 1 kDa and about 35 kDa. Further disclosed is a method of diagnosing and treating a disease.

In certain aspects, the blocking compound is administered at about the same time as the mannosylated carbohydrate polymeric therapeutic and/or diagnostic compound. In alternative aspects, the step of administering the blocking compound is followed by a time interval before the administration of the mannosylated carbohydrate polymeric therapeutic and/or diagnostic compound. According to these embodiments, during this time interval, the blocking compound circulates throughout the body of the subject and binds to off-target CD206 expressing cells in the liver and spleen, or combination thereof to allow for subsequent competitive exclusion of the mannosylated carbohydrate polymeric therapeutic and/or diagnostic compound from binding to such cells. In certain aspects, the time interval is at least about ten minutes. In further aspects, the time interval is between 0 minutes and about 60 minutes. In further aspects, the time interval is from 10 minutes to about 30 minutes. In still further aspects, the time interval is from 10 minutes to about 20 minutes.

In additional embodiments, the blocking compound may be administered simultaneously or shortly before administration of the mannosylated carbohydrate polymeric therapeutic and/or diagnostic compound. The competing blocking compound can be administered at a molar excess to the dose of the mannosylated carbohydrate polymeric therapeutic and/or diagnostic compound. In this case, the blocking agent (i.e. competitor) would freely bind to CD206 expressed on Kupffer cells of the liver or macrophages of the spleen because of the limited barriers between the blood flow and these cells types. In some aspects, the blocking compound preferentially binds to C-type lectin receptor expressing cells in the liver and/or spleen. This competition would prevent or limit the ability of the mannosylated carbohydrate polymer carrying the small drug payload or imaging moiety from binding to these cells.

In some aspects, the administration of the blocking compound results in higher localization of the mannosylated carbohydrate polymeric therapeutic and/or diagnostic compound to a desired target other than the liver and/or spleen compared to without the administration of the blocking compound. In some cases, the desired targets include, but are not limited to, the kidneys, large intestine, and/or skin. Provided the increased target specificity due to the use of the blocking compound, the effective dose of the mannosylated dextran therapeutic and/or diagnostic compound is lower than the effective does of the mannosylated dextran therapeutic and/or diagnostic compound without administration of the blocking compound.

According to certain embodiments of the disclosed methods, the mannosylated carbohydrate polymeric therapeutic and/or diagnostic compound comprises at least one therapeutic moiety. In other embodiments, the mannosylated carbohydrate polymeric therapeutic and/or diagnostic compound comprises at least one diagnostic moiety. In certain aspects, the compound is administered in a therapeutically effective amount. In further aspects, the compound is administered in a prophylactically effective amount.

In certain aspects, the method further comprises administering the composition as a bolus and/or at regular intervals. In certain aspects, the disclosed method further comprises administering the composition intravenously, intraperitoneally, intramuscularly, orally, subcutaneously, intraocularly, intra-tumor injection or transdermally or delivered directly to tumor organ by invasive techniques.

In embodiments, the blocking compound may be administered intravenously as continuous infusions to ensure there is always a sufficient amount of the competitor or blocking compound to occupy the continuously-replenished CD206 receptors in the off-target organs while the mannosylated carbohydrate polymeric therapeutic and/or diagnostic compounds remain in the blood circulation. In some embodiments, the infusion of the blocking compound may continue for an interval of between about 10 minutes to about 1 hour, between about 15 minutes and 45 minutes, or about 20 minutes to 40 minutes after the mannosylated carbohydrate polymeric therapeutic and/or diagnostic compound has been injected intravenously.

The methods provided herein may be practiced in an adjuvant setting. In some embodiments, the method is practiced in a neoadjuvant setting, i.e., the method may be carried out before the primary/definitive therapy. In some embodiments, the method is used to treat an individual who has previously been treated. Any of the methods of treatment provided herein may be used to treat an individual who has not previously been treated. In some embodiments, the method is used as a first line therapy. In some embodiments, the method is used as a second line therapy.

According to certain aspects, the subject suffers a disease that affects the kidneys, large intestine, skin, or a combination thereof. The disease may include, but is not limited to, renal failure, polycystic kidney disease, nephritis, Chron's Disease, irritable bowel syndrome, Celiac disease, colon cancer, psoriasis, diabetic neuropathy, or a combination thereof. Additional diseases that affect the kidneys, large intestine, and skin may be further diagnosed or treated utilizing the methods disclosed.

According to certain alternative embodiments, the subject has been diagnosed with a disease associated with elevated levels of CD206+ macrophages and/or MDSC. Such diseases or conditions include, but are not limited to: acquired immune deficiency syndrome (AIDS), acute disseminated encephalomyelitis (ADEM), Addison's disease, agammaglobulinemia, allergic diseases, alopecia areata, Alzheimer's disease, amyotrophic lateral sclerosis, ankylosing spondylitis, antiphospholipid syndrome, antisynthetase syndrome, arterial plaque disorder, asthma, atherosclerosis, atopic allergy, atopic dermatitis, autoimmune aplastic anemia, autoimmune cardiomyopathy, autoimmune enteropathy, autoimmune hemolytic anemia, autoimmune hepatitis, autoimmune hypothyroidism, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticarial, autoimmune uveitis, Balo disease/Balo concentric sclerosis, Behcet's disease, Berger's disease, Bickerstaffs encephalitis, Blau syndrome, bullous pemphigoid, Castleman disease, celiac disease, Chagas disease, chronic inflammatory demyelinating polyneuropathy, chronic recurrent multifocal osteomyelitis, chronic obstructive pulmonary disease, chronic venous stasis ulcers, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's Syndrome, cutaneous leukocytoclastic angiitis, Degos disease, Dercum's disease, dermatitis herpetiformis, dermatomyositis, Diabetes mellitus type I, Diabetes mellitus type II diffuse cutaneous systemic sclerosis, Dressler's syndrome, drug-induced lupus, discoid lupus erythematosus, eczema, emphysema, endometriosis, enthesitis-related arthritis, eosinophilic fasciitis, eosinophilic gastroenteritis, eosinophilic pneumonia, epidermolysis bullosa acquisita, erythema nodosum, erythroblastosis fetalis, essential mixed cryoglobulinemia, Evans syndrome, fibrodysplasia ossificans progressive, fibrosing alveolitis (or idiopathic pulmonary fibrosis), gastritis, gastrointestinal pemphigoid, Gaucher's disease, glomerulonephritis, Goodpasture's syndrome, Graves' disease, Guillain-Barré syndrome (GBS), Hashimoto's encephalopathy, Hashimoto's thyroiditis, heart disease, Henoch-Schonlein purpura, herpes gestationis (aka gestational pemphigoid), hidradenitis suppurativa, HIV infection, Hughes-Stovin syndrome, hypogammaglobulinemia, infectious diseases (including bacterial infectious diseases), idiopathic inflammatory demyelinating diseases, idiopathic pulmonary fibrosis, idiopathic thrombocytopenic purpura, IgA nephropathy, inclusion body myositis, inflammatory arthritis, inflammatory bowel disease, inflammatory dementia, interstitial cystitis, interstitial pneumonitis, juvenile idiopathic arthritis (aka juvenile rheumatoid arthritis), Kawasaki's disease, Lambert-Eaton myasthenic syndrome, leukocytoclastic vasculitis, lichen planus, lichen sclerosus, linear IgA disease (LAD), lupoid hepatitis (aka autoimmune hepatitis), lupus erythematosus, lymphomatoid granulomatosis, Majeed syndrome, malignancies including cancers (e.g., sarcoma, Kaposi's sarcoma, lymphoma, leukemia, carcinoma and melanoma), Meniere's disease, microscopic polyangiitis, Miller-Fisher syndrome, mixed connective tissue disease, morphea, Mucha-Habermann disease (aka Pityriasis lichenoides et varioliformis acuta), multiple sclerosis, myasthenia gravis, myositis, narcolepsy, neuromyelitis optica (aka Devic's disease), neuromyotonia, occular cicatricial pemphigoid, opsoclonus myoclonus syndrome, Ord's thyroiditis, palindromic rheumatism, PANDAS (pediatric autoimmune neuropsychiatric disorders associated with streptococcus), paraneoplastic cerebellar degeneration, Parkinsonian disorders, paroxysmal nocturnal hemoglobinuria (PNH), Parry Romberg syndrome, Parsonage-Turner syndrome, pars planitis, pemphigus vulgaris, peripheral artery disease, pernicious anaemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restenosis, restless leg syndrome, retroperitoneal fibrosis, rheumatoid arthritis, rheumatic fever, sarcoidosis, schizophrenia, Schmidt syndrome, Schnitzler syndrome, scleritis, scleroderma, sepsis, serum Sickness, Sjögren's syndrome, spondyloarthropathy, Still's disease (adult onset), stiff person syndrome, stroke, subacute bacterial endocarditis (SBE), Susac's syndrome, Sweet's syndrome, Sydenham chorea, sympathetic ophthalmia, systemic lupus erythematosus, Takayasu's arteritis, temporal arteritis (aka “giant cell arteritis”), thrombocytopenia, Tolosa-Hunt syndrome,) transplant (e.g., heart/lung transplants) rejection reactions, transverse myelitis, tuberculosis, ulcerative colitis, undifferentiated connective tissue disease, undifferentiated spondyloarthropathy, urticarial vasculitis, vasculitis, vitiligo, and Wegener's granulomatosis.

### Kits

Also provided herein are kits of pharmaceutical formulations containing the disclosed compounds or compositions. The kits may be organized to indicate a single formulation or combination of formulations. The composition may be sub-divided to contain appropriate quantities of the compound. The unit dosage can be packaged compositions such as packeted powders, vials, ampoules, prefilled syringes or sachets containing liquids.

The compound or composition described herein may be a single dose or for continuous or periodic discontinuous administration. For continuous administration, a kit may include the compound in each dosage unit. For periodic discontinuation, the kit may include placebos during periods when the compound is not delivered. When varying concentrations of the composition, the components of the composition, or relative ratios of the compound or other agents within a composition over time is desired, a kit may contain a sequence of dosage units.

The kit may contain packaging or a container with the compound formulated for the desired delivery route. The kit may also contain dosing instructions, an insert regarding the compound, instructions for monitoring circulating levels of the compound, or combinations thereof. Materials for performing using the compound may further be included and include, without limitation, reagents, well plates, containers, markers or labels, and the like. Such kits are packaged in a manner suitable for treatment of a desired indication. Other suitable components to include in such kits will be readily apparent to one of skill in the art, taking into consideration the desired indication and the delivery route. The kits also may include, or be packaged with, instruments for assisting with the injection/administration or placement of the compound within the body of the subject. Such instruments include, without limitation, an inhalant, syringe, pipette, forceps, measuring spoon, eye dropper or any such medically approved delivery means. Other instrumentation may include a device that permits reading or monitoring reactions in vitro.

The compound or composition of these kits also may be provided in dried, lyophilized, or liquid forms. When reagents or components are provided as a dried form, reconstitution generally is by the addition of a solvent. The solvent may be provided in another packaging means and may be selected by one skilled in the art.

A number of packages or kits are known to those skilled in the art for dispensing pharmaceutical agents. In one embodiment, the package is a labeled blister package, dial dispenser package, or bottle.

In certain aspects, the kit disclosed herein includes a blocking compound comprising a backbone and one or more C-type lectin receptor targeting moieties attached thereto; a mannosylated dextran therapeutic and/or diagnostic compound comprising a carbohydrate polymeric backbone, one or more C-type lectin receptor targeting moieties, and one or more therapeutic and/or diagnostic agents attached thereto; and where the molecular weight of the blocking composition backbone is between about 1 kDa and about 35 kDa. In certain aspects, the kit includes a mannosylated dextran therapeutic and/or diagnostic compound comprising a compound of Formula (I):

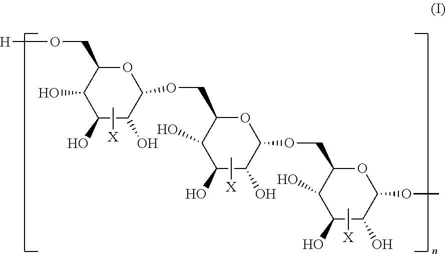

wherein
each X is independently H, L1-A, L1-Y-A, L2-R, or L3-Y, wherein each X is bound to any OH group;
each L1, L2, and L3 are independently leashes;
each A independently comprises a therapeutic agent, a diagnostic agent, a theranostic agent, or H;
each Y independently comprises a chelating agent;
each R independently comprises a mannose-binding C-type lectin receptor targeting moiety or H;
and n is an integer greater than zero, wherein each unit of n may be the same or different; and
wherein at least one R comprises a mannose-binding C-type lectin receptor targeting moiety selected from the group consisting of mannose, fucose, and n-acetylglucosamine; and
wherein at least one A comprises a therapeutic agent, diagnostic agent, or theranostic agent.

In further aspects, the kit includes a blocking compound comprising a compound of Formula (II):

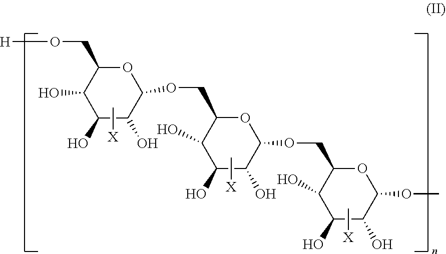

wherein
each X is independently H or L2-R, wherein each X is bound to any OH group;
each R independently comprises a mannose-binding C-type lectin receptor targeting moiety or H;
each L2 is independently a leash;
n is an integer greater than zero, wherein each unit of n may be the same or different; and
wherein at least one R comprises a mannose-binding C-type lectin receptor targeting moiety selected from the group consisting of mannose, fucose, and n-acetylglucosamine.

All publications and patent applications in this specification are indicative of the level of ordinary skill in the art to which this disclosure pertains. All publications and patent applications are herein incorporated by reference to the same extent as if each individual publication or patent application was specifically and individually indicated as incorporated by reference.

### Example

The following example is put forth so as to provide those of ordinary skill in the art with a complete disclosure and description of certain examples of how the compounds, compositions, articles, devices and/or methods claimed herein are made and evaluated, and are intended to be purely exemplary of the disclosure and are not intended to limit the scope of what the inventors regard as their disclosure. However, those of skill in the art should, in light of the present disclosure, appreciate that many changes can be made in the specific embodiments which are disclosed and still obtain a like or similar result without departing from the spirit and scope of the disclosure.

An experiment was performed evaluating the localization of two mannosylated amine dextran (MAD) imaging agents in Balb/c mice with CT26 syngeneic tumors. Both imaging agents conjugated (i.e., carried) the chelating agent dodecane tetraacetic acid (DOTA). DOTA allowed the MAD imaging agents to be efficiently labeled with ⁶⁸gallium ([68]Ga). Labeling with [68]Ga permitted the MAD constructs to be used as PET imaging agents for PET imaging or in conjunction with x-ray based computed tomography (CT) for PET/CT imaging. The two imaging agents differed in size, with one being built on a 3.5 kDa dextran backbone (MAN-DOTA-3.5) and had a final calculated molecular weight (Mw) of 8.7 kDa, while the second was built on a 10 kDa backbone (MAN-DOTA-10) and had a final calculated molecular weight (Mw) of 22.6 kDa.

In the experiment, 5 μg of either MAD-DOTA-3.5 and MAD-DOTA-10 were labeled with approximately 100 μCi of [68]Ga and injected intravenously (IV) into anesthetized tumor baring mice. The animals then underwent dynamic PET/CT imaging for 90 minutes to visualize the localization of the imaging agents to the tumors and to various organs including the liver. At the end of the dynamic imaging, the animals were euthanized and necropsied to remove the tumors and various organs. The extracted tissues were weighed and the amount of retained radioactivity in each organ was determined using an automated gamma counter (Hidex). The data were decay corrected to time of sacrifice and used to calculate the percent injected dose per gram of tissue (% ID/g). In some experimental arms, the animals were injected IV with 250 μg of an unlabeled MAD construct (“unlabeled MAN-DOTA”; i.e., blocking compound disclosed herein) 5 minutes prior to their injections of the [68]Ga labeled imaging agents (“[68]Ga MAN-DOTA”; i.e., mannosylated carbohydrate polymeric diagnostic compound disclosed herein).

The experiment had several parts and was designed to evaluate a hypothesis that a high molecular weight (HMW) unlabeled MAD construct (Mw≈300 kDa) could competitively block the localizations of [68]Ga labeled MAD-DOTA-3.5 and [68]Ga MAD-DOTA-10 to CD206 expressed by Kupffer cells of the liver without simultaneously diminishing the localization of the imaging agents to tumor associated macrophages (TAMs) in the CT26 tumors. This hypothesis was based on the premise that the HMW construct could bind to CD206 expressed on Kupffer cells as Kupffer cells are directly exposed to the blood flow, however, due to its large size, the HMW construct would have a reduced ability to exit the blood flow and penetrate into tumors.

This would permit the smaller MAD constructs to enter the tumors and bind to CD206 on TAMs without competition from the HMW construct. This hypothesis regarding the HMW constructs has been evaluated previously under U.S. application Ser. No. 17/039,409, which has been incorporated herein by reference in its entirety. However, this Example further evaluates the effects of unlabeled MAN-DOTA as a blocking compound.

Data from 6 arms or treatment groups (n=4 mice/treatment group) evaluated were provided. In this Example, the mice were injected only with the [68]Ga MAN-DOTA construct or injected with the [68]Ga MAN-DOTA constructs 5 minutes after they had been injected with 250 μg of the unlabeled MAN-DOTA construct as described in Table 1. The 250 μg of the unlabeled MAN-DOTA was a 50-fold (50×) molar excess relative to the 5 μg of labeled [68]Ga MAN-DOTA.

TABLE 1
Balb/c mice with CT26 Tumors

| Treatment group | | |
|---|---|---|
| (n = 4 mice/ | Imaging Agent | Blocking Compound |
| treatment) | (5 μg) | (250 μg) |
| 1 | [68]Ga-MAN-DOTA-3.5 | None (Tumor Bearing) |
| 2 | [68]Ga-MAN-DOTA-3.5 | Unlabeled MAN-DOTA- |
| | | 3.5 |
| 3 | [68]Ga-MAN-DOTA-3.5 | HMW Construct |
| 4 | [68]Ga-MAN-DOTA-10 | None (Tumor Bearing) |
| 5 | [68]Ga-MAN-DOTA-10 | Unlabeled MAN-DOTA-10 |
| 6 | [68]Ga-MAN-DOTA-10 | HMW Construct |

Hypothesis: The hypothesized results were that the Imaging Agents would localize to varying degrees to all tissues and organs (with the exception of the brain due to exclusion via the blood-brain barrier), because CD206 expressing macrophages reside in all tissues of the body. The amount of localization would vary directly with the number or density of tissue resident CD206 expressing macrophages and inversely with the strength of barriers limiting the ability of the imaging agents to exit the blood and penetrate into tissues. Since the Kupffer cells of the liver are highly numerous and directly exposed to the blood flow (i.e., no barriers for exiting the blood flow and penetrating into tissues where CD206 macrophages reside), it was expected that much of the labeled MAN-DOTA constructs would localize to the liver. It was further hypothesized that competition with the 50× unlabeled MAN-DOTA would greatly suppress liver (i.e., Kupffer cell) localization. It was also hypothesized that competition with 50× unlabeled MAN-DOTA construct would suppress localization of [68]Ga-MAN-DOTA in all tissues, but perhaps to varying degrees based on the strength of the barriers for exiting the blood flow and penetrating into tissues for both the imaging agent and the unlabeled MAN-DOTA. The HMW competitor was hypothesized to greatly suppress [68]Ga-MAN-DOTA localization to the liver (i.e., Kupffer cells) but would likely have limited impact on the localization of the labeled constructs to tissues because its size would limit its ability to exit the blood flow and compete with the smaller labeled constructs for binding to CD206 on tissue resident macrophages.

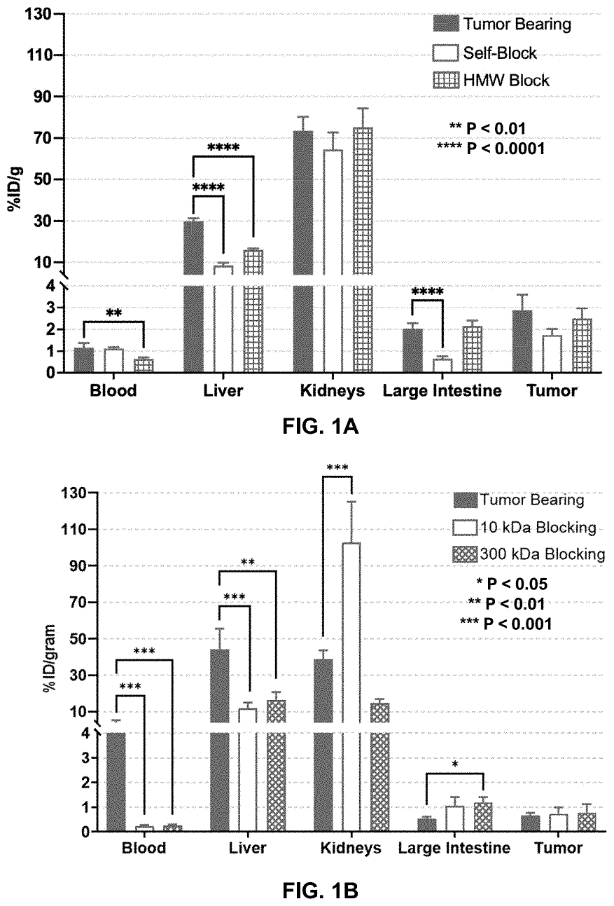

Observed Results: The results are shown in Tables 2A, 2B, 3A, and 3B (observed % ID/g), with selected results graphically presented in FIG. 1A and FIG. 1B. First, competition with either the unlabeled MAN-DOTA (self-competition) or the HMW construct dramatically and significantly reduced localization of the [68]Ga labeled imaging agents to the liver. However, the results for several of the other organs and tissue deviated from the hypothesis. For the [68]Ga-MAN-DOTA-3.5 studies, all of the organ specific % ID/g of tissue determinations were reduced by competition, but the degree of reduction varied dramatically. Many organs followed a localization pattern similar to the liver with dramatic and significant reductions in localization with competition with the unlabeled MAN-DOTA. However, [68]Ga-MAN-DOTA-3.5 localization to the spleen (22.5% reduction) and kidneys (12.4% reduction) were modest and non-significant. A statistic may be calculated by comparing the % ID/g localization to the liver relative to the % ID/g localization to another organ as % ID/g (other organ) divided by the % ID/g (liver). For the spleen and [68]Ga-MAN-DOTA-3.5, this ratio is 0.39 for the imaging agent by itself and 1.07 when localization is self-competed with the unlabeled MAN-DOTA. This represents a 273% increase in relative localization associated with self-competition. For the kidneys, these ratios are 2.46 and 7.61 for the no competition and self-competition data sets respectively, representing a 309% increase in relative localization associated with self-competition. These were unanticipated and surprising results.

TABLE 2A

| | [⁶⁸Ga]3.5 - Control | | | [⁶⁸Ga]3.5 - Tumor Bearing | | |
|---|---|---|---|---|---|---|
| | Average | | StDev | Average | | StDev |
| Blood | 0.694 | +/− | 0.080 | 1.167 | +/− | 0.207 |
| Heart | 3.298 | +/− | 0.199 | 3.210 | +/− | 0.198 |
| Lungs | 2.228 | +/− | 0.181 | 2.522 | +/− | 0.097 |
| Spleen | 16.267 | +/− | 6.516 | 11.742 | +/− | 2.068 |
| Liver | 32.398 | +/− | 4.966 | 29.852 | +/− | 1.484 |
| Kidneys | 65.441 | +/− | 3.641 | 73.512 | +/− | 6.783 |
| S. Intestines | 1.691 | +/− | 0.439 | 2.059 | +/− | 0.228 |
| L. Intestines | 1.781 | +/− | 0.187 | 2.034 | +/− | 0.247 |
| Skin | 0.968 | +/− | 0.145 | 1.097 | +/− | 0.199 |
| Muscle | 0.455 | +/− | 0.116 | 0.430 | +/− | 0.155 |
| Bone | 2.054 | +/− | 0.546 | 2.469 | +/− | 0.352 |
| Brain | 0.079 | +/− | 0.028 | 0.100 | +/− | 0.031 |
| Tumor | — | +/− | — | 2.872 | +/− | 0.725 |

TABLE 2B

| | [⁶⁸Ga]3.5 w/3.5 | | | [⁶⁸Ga]3.5 w/300 | | |
|---|---|---|---|---|---|---|
| | kDa Blocking | | | kDa Blocking | | |
| | Average | | StDev | Average | | StDev |
| Blood | 1.126 | +/− | 0.066 | 0.639 | +/− | 0.068 |
| Heart | 1.119 | +/− | 0.150 | 3.327 | +/− | 0.672 |
| Lungs | 1.640 | +/− | 0.214 | 2.912 | +/− | 0.430 |
| Spleen | 9.096 | +/− | 1.167 | 9.161 | +/− | 1.825 |
| Liver | 8.468 | +/− | 1.377 | 16.086 | +/− | 0.682 |
| Kidneys | 64.424 | +/− | 8.297 | 75.293 | +/− | 9.003 |
| S. Intestines | 0.785 | +/− | 0.287 | 2.035 | +/− | 0.102 |
| L. Intestines | 0.647 | +/− | 0.106 | 2.161 | +/− | 0.242 |
| Skin | 0.606 | +/− | 0.049 | 1.029 | +/− | 0.242 |
| Muscle | 0.190 | +/− | 0.039 | 0.450 | +/− | 0.090 |
| Bone | 1.383 | +/− | 0.522 | 1.574 | +/− | 0.488 |
| Brain | 0.060 | +/− | 0.010 | 0.120 | +/− | 0.036 |
| Tumor | 1.740 | +/− | 0.283 | 2.499 | +/− | 0.468 |

TABLE 3A

| | [⁶⁸Ga]10 - Control | | | [⁶⁸Ga]10 - Tumor Bearing | | |
|---|---|---|---|---|---|---|
| | Average | | StDev | Average | | StDev |
| Blood | 4.122 | +/− | 0.939 | 3.989 | +/− | 1.294 |
| Heart | 2.408 | +/− | 0.365 | 1.954 | +/− | 0.145 |
| Lungs | 2.262 | +/− | 0.485 | 1.559 | +/− | 0.152 |
| Spleen | 17.154 | +/− | 6.463 | 18.884 | +/− | 6.949 |
| Liver | 53.883 | +/− | 12.577 | 44.384 | +/− | 11.208 |
| Kidneys | 50.918 | +/− | 14.340 | 38.692 | +/− | 5.007 |
| S. Intestines | 2.141 | +/− | 0.354 | 1.525 | +/− | 0.208 |
| L. Intestines | 0.785 | +/− | 0.135 | 0.549 | +/− | 0.073 |
| Skin | 0.488 | +/− | 0.174 | 0.290 | +/− | 0.063 |
| Muscle | 0.143 | +/− | 0.020 | 0.164 | +/− | 0.030 |
| Bone | 2.957 | +/− | 0.418 | 2.523 | +/− | 0.407 |
| Brain | 0.382 | +/− | 0.275 | 0.106 | +/− | 0.014 |
| Tumor | — | +/− | — | 0.664 | +/− | 0.111 |

TABLE 3B

| | [⁶⁸Ga]10 w/10 | | | [⁶⁸Ga]10 w/300 | | |
|---|---|---|---|---|---|---|
| | kDa Blocking | | | kDa Blocking | | |
| | Average | | StDev | Average | | StDev |
| Blood | 0.226 | +/− | 0.051 | 0.245 | +/− | 0.051 |
| Heart | 1.308 | +/− | 0.278 | 3.374 | +/− | 0.520 |
| Lungs | 1.316 | +/− | 0.218 | 2.248 | +/− | 0.133 |
| Spleen | 3.981 | +/− | 1.199 | 24.014 | +/− | 4.442 |
| Liver | 11.859 | +/− | 3.067 | 16.279 | +/− | 4.495 |
| Kidneys | 102.799 | +/− | 22.317 | 14.728 | +/− | 2.308 |
| S. Intestines | 1.843 | +/− | 0.494 | 2.461 | +/− | 0.454 |
| L. Intestines | 1.058 | +/− | 0.355 | 1.199 | +/− | 0.214 |
| Skin | 0.913 | +/− | 0.331 | 0.611 | +/− | 0.077 |
| Muscle | 0.160 | +/− | 0.050 | 0.193 | +/− | 0.052 |
| Bone | 1.785 | +/− | 0.484 | 3.826 | +/− | 0.836 |
| Brain | 0.054 | +/− | 0.019 | 0.086 | +/− | 0.011 |
| Tumor | 0.735 | +/− | 0.260 | 0.783 | +/− | 0.336 |

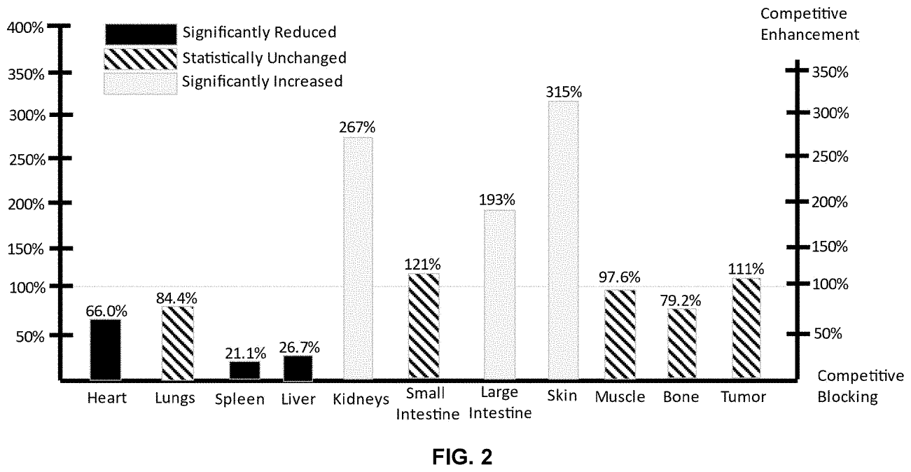

The results from the imaging studies evaluating the [68]Ga-MAN-DOTA-10 construct with and without competitive blocking are shown in FIG. 2, and were equally, if not more surprising. First, unlike the results with the [68]Ga-MAN-DOTA-3.5 imaging studies, self-competition with the Unlabeled MAN-DOTA resulted in a dramatic and significant reduction in imaging agent localization to the spleen. In this case, the ratio of % ID/g (spleen) divided by the % ID/g (liver) went down from 0.43 without competition to 0.34 with Unlabeled MAN-DOTA self-competition, a relative decrease in localization of 21.1%. The exact opposite effect was observed for the [68]Ga-MAN-DOTA-10 construct localization to the kidneys. The % ID/g for the kidneys increased from 38.69 without self-competition to 102.80 with self-competition, a highly significant increase (266%). Relative to liver localization, without self-competition, the ratio of [68]Ga-MAN-DOTA-10 localization to the kidneys (% ID/g) divided by its localization (% ID/g) to the liver was 0.87. With self-competition, this ratio increased very significantly to 8.67, a very dramatic increase of 994% or nearly a 10-fold increase. In the previous result with [68]Ga-MAN-DOTA-3.5, the kidney to liver ratio returned a 309% increase with self-competition. This change is in the same direction but less than a third the magnitude of the ratio change observed for the [68]Ga-MAN-DOTA-10 construct (974%). The imaging agent localization results for the large intestine and skin using the [68]Ga-MAN-DOTA-10 construct with and without self-competition were also similar to what was observed for the kidneys. Without self-competition, the % ID/g localization for the large intestine and skin were 0.549 and 0.290, respectively. With self-competition, the localizations increased to 1.058% ID/g (193%) and 0.913% ID/g (315%), respectively. When evaluated for changes in localization relative to the liver, the ratio of organ % ID/g divided by liver % ID/g for the large intestine and skin went from 0.0124 and 0.0065 respectively without self-competition to 0.089 (721%) and 0.077 (1178%) with self-competition. While the amount of [68]Ga-MAN-DOTA-10 construct localization (% ID/g) in the large intestine and skin were relatively low without self-competition, the relative increases in localizations compared to the liver with self-competition were striking and surprisingly large.

The results showing comparative data analysis with self competition are further summarized in Tables 4A and 4B below.

TABLE 4A
Part A: [68]Ga-MAN-DOTA-3.5

| | | Liver | | Liver | | % Change |
|---|---|---|---|---|---|---|
| | % ID/g | ratio | % ID/g | ratio | % ratio | in Liver |
| Organ | NC* | NC | SC\grave{ } | SC | SC/NC | Ratio |
| Liver | 29.852 | N/A | 8.468 | N/A | 28.4% | N/A |
| Spleen | 11.742 | 0.393 | 9.096 | 1.074 | 77.5% | 273% |
| Kidneys | 73.512 | 2.463 | 64.424 | 7.608 | 87.6% | 309% |

TABLE 4B
Part B: [68]Ga-MAN-DOTA-10

| | | Liver | | Liver | | % Change |
|---|---|---|---|---|---|---|
| | % ID/g | ratio | % ID/g | ratio | % ratio | in Liver |
| Organ | NC* | NC | SC\grave{ } | SC | NC/SC | Ratio |
| Liver | 44.384 | N/A | 11.859 | N/A | 26.7% | N/A |
| Spleen | 18.884 | 0.425 | 3.981 | 0.336 | 21.1% | 79% |
| Kidneys | 38.692 | 87.2% | 102.799 | 8.67 | 265.7% | 994% |
| Lg. Intest. | 0.549 | 0.012 | 1.058 | 0.089 | 192.7% | 721% |
| Skin | 0.290 | 0.0065 | 0.913 | 0.077 | 315% | 1178% |

*NC=No self competition
′SC=Self competition (unlabeled MAN-DOTA)

Liver Ratio=((Organ % ID/g)/(Liver % ID/g))×100

% ratio=NC/SC×100

Discussion: The results demonstrate that the administration of [68]Ga-MAN-DOTA-3.5 or [68]Ga-MAN-DOTA-10 following an IV administration of a 50-fold molar excess of Unlabeled MAN-DOTA (self-competition) resulted in a dramatic and highly significant reduction in localization of the labeled imaging agents to the Kupffer cells of the liver. This followed the hypothesized result. It was also hypothesized that self-competition would reduce labeled imaging agent localization to other organs, perhaps to varying degrees based on the strength of barriers to the labeled or unlabeled imaging agents exiting the blood flow and penetrating into tissues or tumors. However, it was unexpected that self-competition would result in increases in the localization of labeled imaging agent in specific organs or tissues. This hypothesis was not met for at least three organs injected with [68]Ga-MAN-DOTA-10: the kidneys, large intestine, and skin. In these three organs, self-competition resulted in increased localization of the labeled [68]Ga-MAN-DOTA-10. This was a highly unexpected result.

As the DOTA chelator does not contribute to the binding of the MAD constructs to CD206, the chelator does not contribute to the competitive blocking of the labeled, [68]Ga-MAN-DOTA constructs by the unlabeled competitor. Therefore, the competitive blocking construct would function equally well as a competitor if it lacked the DOTA chelator. Furthermore, the surprising result that competitive blocking of [68]Ga-MAN-DOTA-10 by unlabeled MAN-DOTA-10 concurrently reduced localization of [68]Ga-MAN-DOTA-10 to the liver while increasing localization to the kidneys, would have utility for improving imaging studies of the kidneys. Therefore, further provided within this disclosure are methods for improving imaging of the kidneys comprising the steps of administering the blocking compound as described throughout the disclosure, and the mannosylated carbohydrate polymeric therapeutic and/or diagnostic compound as described throughout the disclosure, to a subject in need thereof.

Without being limited to any particular mechanism or theory, this effect (i.e., increased localization with self-competition) is likely dependent on the molecular weight of the imaging agent. The solution may lie in the interaction of two variables and a constant. The first variable involves the molecular weight of the imaging agents. In this Example, all mice were injected with 5 μg of labeled imaging agent and, in the self-competition groups, with 250 μg of the unlabeled imaging agent. However, because MAN-DOTA-3.5 had a molecular weight of 8.7 kDa and MAN-DOTA-10 had a molecular weight of 22.6 kDa, animals injected with MAN-DOTA-3.5 received more than twice as many molecules of the imaging agent as did the animals injected with MAN-DOTA-10. The constant value in this interaction is the number of CD206 molecules on Kupffer cells of the liver, which is fixed. Each CD206 molecule will bind one MAD construct molecule. After all of the CD206 molecules have bound a MAD construct molecule, no more CD206 molecules can bind as the receptors are saturated. When the self-competitor is injected, it can saturate the CD206 on the Kupffer cells at which point the remaining self-competitor remains in circulation, extending its blood half-life. This is also true for the labeled imaging agent-after the CD206 on Kupffer cells have been saturated, the blood half-life of the labeled imaging agent is extended because it is not being bound up in the liver. The extended blood half-life of the Unlabeled MAN-DOTA provides it with a greater opportunity to compete with the labeled imaging agent in the other tissues. Similarly, the number of labeled imaging agent molecules in circulation was increased with a longer blood-half life because the labeled imaging agent was not being sequestered in Kupffer cells.

The second variable is the number of CD206 receptors in the organ. Once these receptors have been saturated, no more localization can occur.

In the cases of the kidneys, large intestine, and skin, without being limited to any particular mechanism or theory, a larger portion of the MAN-DOTA-10 self-competitor is likely localizing to the Kupffer cells than is the case for MAN-DOTA-3.5 because there are twice as many MAN-DOTA-3.5 molecules and the number of Kupffer cells is fixed. When the labeled [68]Ga-MAN-DOTA-10 is administered after the self-competitor, it stays in circulation longer at a higher concentration while the unlabeled self-competitor is relatively depleted because it mostly bound to the Kupffer cells. With higher systemic concentrations of [68]Ga-MAN-DOTA-10 and lower concentrations of self-competitors, more of the imaging agent binds to these organs. In other organs, the change in the organ/liver ratios indicate that a similar process is in effect but is not sufficient to bring the localization to a point greater than what was observed without self-competition.

With regard to the present disclosure, the above identified observations allow for greater delivery of imaging agents and therapeutics to the kidneys, large intestine, and skin while simultaneously reducing off target exposure to the liver and other organs.

Although the present disclosure has been described with reference to preferred embodiments, persons skilled in the art will recognize that changes may be made in form and detail without departing from the spirit and scope of the disclosure.

What is claimed is:

1. A method of increasing target specificity of a mannosylated carbohydrate polymeric therapeutic and/or diagnostic compound comprising:
a. administering a blocking compound comprising a backbone and one or more C-type lectin receptor targeting moieties attached thereto; and
b. administering an effective amount of the mannosylated carbohydrate polymeric therapeutic and/or diagnostic compound comprising a carbohydrate polymeric backbone, one or more C-type lectin receptor targeting moieties, and one or more therapeutic, diagnostic, or theranostic agents attached thereto;
wherein the blocking compound backbone is between about 1 kDa and about 35 kDa.

2. The method of claim 1, wherein the mannosylated carbohydrate polymeric therapeutic and/or diagnostic compound comprises the compound of Formula (I): 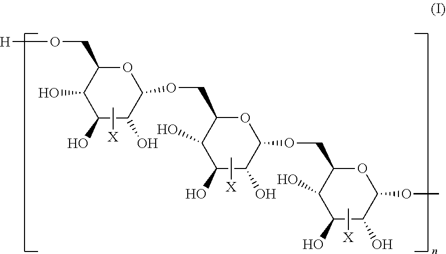
wherein
each X is independently H, L1-A, L1-Y-A, L2-R, or L3-Y, wherein each X is bound to any OH group;
each L1, L2, and L3 are independently leashes having the formula —(CH₂)ₚS(CH₂)_{q}—NH—, wherein p and q are independently integers from 0 to 5;
each A independently comprises a therapeutic agent, a diagnostic agent, a theranostic agent, or H;
each Y independently comprises a chelating agent;
each R independently comprises a mannose-binding C-type lectin receptor targeting moiety or H;
and n is an integer greater than zero, wherein each unit of n may be the same or different; and
wherein at least one R comprises a mannose-binding C-type lectin receptor targeting moiety selected from the group consisting of mannose, fucose, and n-acetylglucosamine; and
wherein at least one A comprises a therapeutic agent, diagnostic agent, or theranostic agent.

3. The method of claim 1, wherein the carbohydrate polymeric backbone is selected from the group consisting of dextran, cellulose, polyethylene glycol, and polypeptides.

4. The method of claim 1, wherein the one or more C-type lectin receptor targeting moieties is attached to the blocking compound backbone via a leash having the formula (CH₂)ₚS(CH₂)_{q}—NH—, wherein p and q are independently integers from 0 to 5.

5. The method of claim 1, wherein the mannosylated dextran therapeutic and/or diagnostic compound dextran backbone is between about 1 kDa and about 50 kDa, and wherein the blocking compound backbone is between about 1 kDa and 30 kDa.

6. The method of claim 1, wherein the blocking compound comprises a compound of Formula (II): 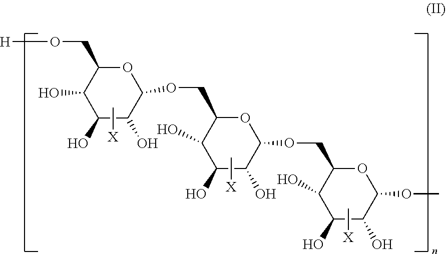
wherein
each X is independently H or L2-R, wherein each X is bound to any OH group;
each R independently comprises a mannose-binding C-type lectin receptor targeting moiety or H;
each L2 is independently a leash having the formula —(CH₂)ₚS(CH₂)_{q}—NH—, wherein p and q are independently integers from 0 to 5;
n is an integer greater than zero, wherein each unit of n may be the same or different; and
wherein at least one R comprises a mannose-binding C-type lectin receptor targeting moiety selected from the group consisting of mannose, fucose, and n-acetylglucosamine.

7. The method of claim 1, wherein the blocking compound does not contain a therapeutic agent, diagnostic agent, theranostic agent, or a detectable moiety.

8. The method of claim 1, wherein the step of administering the blocking compound is followed by a time interval of from zero to about 60 minutes before the step of administering the mannosylated dextran therapeutic and/or diagnostic compound.

9. The method of claim 1, wherein the blocking compound and the mannosylated dextran therapeutic and/or diagnostic compound are administered simultaneously.

10. The method of claim 1, wherein the mannosylated dextran therapeutic and/or diagnostic compound has decreased localization to C-type lectin receptor cells in the liver and/or spleen relative to a subject administered a comparable dose of mannosylated dextran therapeutic and/or diagnostic compound without administration of the blocking compound.

11. The method of claim 1, wherein the administration of the blocking compound results in higher localization to a desired target other than the liver and/or spleen compared to without the administration of the blocking compound.

12. The method of claim 11, wherein the desired target comprises the kidneys, large intestine, and/or skin.

13. The method of claim 1, wherein the effective dose of the mannosylated dextran therapeutic and/or diagnostic compound is lower than the effective does of the mannosylated dextran therapeutic and/or diagnostic compound without administration of the blocking compound.

14. The method of claim 1, wherein the blocking compound preferentially binds to CD206 expressing cells in the liver and/or spleen.

15. The method of claim 1, wherein the subject has been diagnosed with disease associated with the kidneys, large intestine, or skin.

16. The method of claim 15, wherein the disease comprises renal failure, polycystic kidney disease, nephritis, Crohn's Disease, irritable bowel syndrome, Celiac disease, colon cancer, psoriasis, diabetic neuropathy, or a combination thereof.

17. The method of claim 1, wherein the blocking agent is administered to a human subject in an amount of at least about 50 mg.

18. A method of diagnosing and treating a disease comprising:
a. administering a blocking compound comprising a backbone and one or more C-type lectin receptor targeting moieties attached thereto, wherein the blocking compound backbone is from about 1 kDa to about 35 kDa;
b. administering an effective amount of a mannosylated carbohydrate polymeric therapeutic and/or diagnostic compound comprising a carbohydrate polymeric backbone and one or more C-type lectin receptor targeting moieties and one or more therapeutic, diagnostic, or theranostic agents attached thereto,
wherein the administration of the blocking compound increases target specificity of the mannosylated carbohydrate polymeric therapeutic and/or diagnostic compound.

19. The method of claim 18, wherein the disease affects the kidneys, large intestine, or skin.

20. The method of claim 19, wherein the disease comprises renal failure, polycystic kidney disease, nephritis, Chron's Disease, irritable bowel syndrome, Celiac disease, colon cancer, psoriasis, diabetic neuropathy, or a combination thereof.

21. The method of claim 18, wherein the mannosylated carbohydrate polymeric therapeutic and/or diagnostic compound comprises a compound of Formula (I): 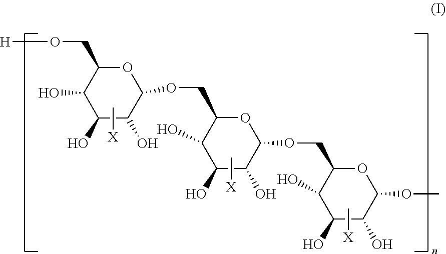
wherein
each X is independently H, L1-A, L1-Y-A, L2-R, or L3-Y, wherein each X is bound to any OH group;
each L1, L2, and L3 are independently leashes having the formula —(CH₂)ₚS(CH₂)_{q}—NH—, wherein p and q are independently integers from 0 to 5;
each A independently comprises a therapeutic agent, a diagnostic agent, a theranostic agent, or H;
each Y independently comprises a chelating agent;
each R independently comprises a mannose-binding C-type lectin receptor targeting moiety or H;
and n is an integer greater than zero, wherein each unit of n may be the same or different; and
wherein at least one R comprises a mannose-binding C-type lectin receptor targeting moiety selected from the group consisting of mannose, fucose, and n-acetylglucosamine; and
wherein at least one A comprises a therapeutic agent, diagnostic agent, or theranostic agent.

22. The method of claim 18, wherein the blocking compound comprises a compound of Formula (II): 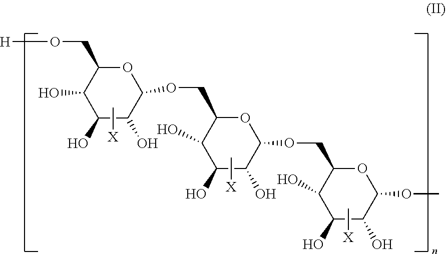
wherein
each X is independently H or L2-R, wherein each X is bound to any OH group;
each R independently comprises a mannose-binding C-type lectin receptor targeting moiety or H;
each L2 is independently a leash having the formula —(CH₂)ₚS(CH₂)_{q}—NH—, wherein p and q are independently integers from 0 to 5;
n is an integer greater than zero, wherein each unit of n may be the same or different; and
wherein at least one R comprises a mannose-binding C-type lectin receptor targeting moiety selected from the group consisting of mannose, fucose, and n-acetylglucosamine.

23. The method of claim 18, wherein the blocking compound backbone is from about 1 kDa to about 50 kDa, and the mannosylated dextran therapeutic and/or diagnostic compound dextran backbone is between about 1 kDa and about 30 kDa.
